(19) United States
(12) Patent Application Publication
(10) Pub. No.: US 2024/0123289 A1
(43) Pub. Date: Apr. 18, 2024

(54) Automated Athletic Evaluation and Training

(71) Applicant: Proteus Motion Inc., Brooklyn, NY (US)

(72) Inventors: Samuel Adam Miller, Mount Pleasant, SC (US); Joseph Williams Waterman, Brooklyn, NY (US); Jason Shaev, Brooklyn, NY (US); William Gabrenya, III, Brooklyn, NY (US)

(21) Appl. No.: 18/485,462

(22) Filed: Oct. 12, 2023

Related U.S. Application Data
(60) Provisional application No. 63/379,253, filed on Oct. 12, 2022.

(51) Int. Cl. A63B 24/00; A63B 71/06

(52) U.S. Cl. CPC A63B 24/0075; A63B 71/0616; A63B 2024/0015; A63B 2024/0068; A63B 2214/00; A63B 2220/30; A63B 2220/40; A63B 2220/803; A63B 2230/60

(57) ABSTRACT

Performance of an athlete is evaluated using position data of the athlete over time during performance of a set of movements. Movement metrics for the set of movements are determined, the movement metrics including measures of acceleration and power. Performance metrics for the athlete are then calculated to indicate the athlete's strength and speed. A reference data set is defined based on various attributes associated with the athlete. The performance metrics are applied to the reference data set to determine a performance category for the athlete, the performance category indicating relative strength and speed of the athlete among other athletes represented in the reference data set. Lastly, a training regimen for the athlete is generated based on the performance category.

### RELATED APPLICATION

This application claims the benefit of U.S. Provisional Application No. 63/379,253, filed on Oct. 12, 2022. The entire teachings of the above application are incorporated herein by reference.

### BACKGROUND

The assessment of an athlete's physical capabilities is fundamental in sports. Conventional measurement of strength and speed has been reliant upon manual tests, such as stopwatch timings for speed, weightlifting measures for strength, or manual observation and assessment of an athlete's performance. Such approaches often introduce human error, are time-consuming, and may lack standardization.

Automated measurement tools can measure athletic performance with greater accuracy than what was previously possible. For example, isokinetic dynamometers can provide a precise measure of applied force throughout a joint range of motion. However, the insights provided by such instruments are limited, leaving an athlete without necessary context for the measurements nor sufficient guidance on how to improve the athlete's performance.

### SUMMARY

Example embodiments include methods of evaluating an athlete. Position data of the athlete during performance of a set of movements may be obtained, the position data indicating position of the athlete over time during the performance of the set of movements. Movement metrics for the set of movements may be determined based on the position data, the movement metrics including measures of acceleration and power. Performance metrics for the athlete may then be calculated based on the movement metrics, the performance metrics including strength and speed. A reference data set may be defined based on a plurality of attributes associated with the athlete. The performance metrics may then be applied to the reference data set to determine a performance category for the athlete, the performance category indicating relative strength and speed of the athlete among other athletes represented in the reference data set. Lastly, a training regimen for the athlete may be generated based on the performance category.

The plurality of attributes associated with the athlete may include at least one of age, gender, weight, sport, height, weight, handedness, position in sport, and skill level of sport. Defining the reference data set may include selecting a subset of a larger data set based on similarities between attributes associated with the subset and the attributes associated with the athlete. A subset of the set of movements that exhibit peak performance may be determined, and the performance metrics may be based on the subset to the exclusion of movements outside of the subset. The performance category may indicate a degree of balance between the relative strength and speed, and generating the training regimen may include selecting a set of exercises for the training regimen that are indicated to improve the degree of balance between strength and speed.

The performance category may be selected from a set of categories including at least one of 1) low strength or low power, 2) speed dominant or acceleration dominant, 3) strength dominant or power dominant, and 4) high strength and speed or balanced strength and speed. The position data may be obtained from a machine applying a resistance opposing the set of movements. Determining the movement metrics for the set of movements may be based on a measured force applied by the athlete during the performance of the set of movements.

The training regimen may include instructions defining one or more individualized training sessions configured based on the performance of the set of movements. The training regimen may include instructions defining at least one workout, training session, or rehabilitation session. The training regimen may also include instructions defining at least one of a resistance level, training equipment, number of repetitions, number of sets, recommended velocity range, and frequency for at least one training movement.

Determining the performance category may be based on a difference between the performance metrics and the reference data set, the difference being at least one or raw values and percentile values. Based on the movement metrics, at least one of 1) an endurance metric indicating a measure of endurance of the athlete, 2) a consistency metric indicating a measure of consistency of movement performed by the athlete, and 3) a range of motion (ROM) metric indicating a measure of mobility or flexibility of the athlete may be determined. Generating the training regimen may include selecting a set of exercises for the training regimen that are indicated to improve at least one of the endurance metric, the consistency metric, and the ROM metric.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIGS. 1A-B are flow diagrams of a process of evaluating athletic performance in one embodiment.

FIG. 2 is a table of results of an evaluation in one embodiment.

FIGS. 3A-B are tables of results of an elasticity evaluation in one embodiment.

FIG. 4 is a table illustrating movements performed by an athlete in one embodiment.

FIG. 5 is a table illustrating movement metrics calculated from position data in one embodiment.

FIG. 6 is a table illustrating percentile scores corresponding to movement data in one embodiment.

FIG. 7 is a table illustrating averaged percentile scores in one embodiment.

FIG. 8 is a table illustrating final percentile scores in one embodiment.

FIG. 9 is a table illustrating athlete performance classification and corresponding training regimen in one embodiment.

FIG. 10 is a flow diagram of a process of evaluating athletic performance in one embodiment.

FIG. 11 is a diagram of a computer network in which example embodiments may be implemented.

FIG. 12 is a diagram of a computer system in which example embodiments may be implemented.

### DETAILED DESCRIPTION

A description of example embodiments follows.

Many athletes and trainers do not yet have the expertise to interpret and take the next best actions informed by the results of a power-based test. These users also do not have a straightforward way to track their progress between tests, using awkward workarounds to compare two reports. Example embodiments, described below, provide a solution for accurate evaluation of an athlete's strength and speed (or acceleration and power), and provide a corresponding training regimen to enable the athlete to improve their performance.

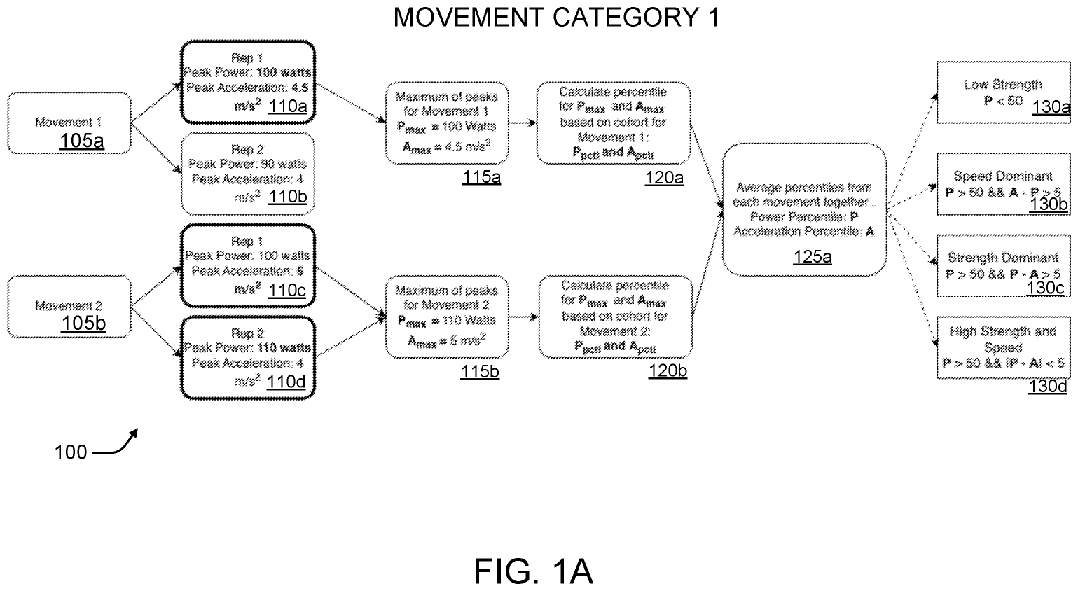

FIGS. 1A-B are flow diagrams of a process 100 of evaluating athletic performance. The process 100 may be operated by a computing device or network as described in further detail below. To obtain data for evaluation, a trainer or an athlete may select a performance test to perform based on their training goals. The performance test (assessment) may be a curated collection of movements, grouped into movement categories, that an athlete performs for the purpose of assessing their physical ability. The performance test may be configured with movements selected to evaluate the athlete's performance in a particular sport (e.g., golf, baseball) or a more general measure of strength, speed, and/or power. Each movement category may be a collection of movements that have similar movement patterns and/or activate similar muscles (e.g., upper body movements, lower body movements, push movements, pull movements, upper body horizontal push, core rotation). FIG. 1A depicts the processing of data of a first movement category, while FIG. 1B depicts the processing of data of a second movement category. Although two movement categories are depicted, example embodiments may process additional data of additional movement categories. Further, each movement may be a specific exercise that that athlete performs with or without resistance (e.g., tricep extension, chest press)

The athlete may perform the test by completing each movement in the test a set number of times (reps) at a set resistance. The athlete may perform the movements in connection with one or more instruments configured to collect position/movement data of the athlete during the performance (105a-h). The instruments may include, for example, isokinetic dynamometers, hand held dynamometers, force plates, markerless motion capture systems, heart rate variability (HRV) wearables, body composition scanners, and cable machines.

For each movement, the movement data 105a-d is processed to determine the maximum power and acceleration the athlete achieved per rep (110a-h). Once the maximum power and acceleration is determined for each rep (110a-h), the maximum power and acceleration the athlete achieved across all reps of that movement, denoted Pₘₐₓ and Aₘₐₓ, is determined (115a-d). Pₘₐₓ and Aₘₐₓ may be from different reps of each movement. Pₘₐₓ and Aₘₐₓ may then be converted into percentiles, denoted P_{pctl} and A_{pctl}, by comparing the values against historical results from other users in their cohort who have completed the same movements at the same resistance (120a-d). The cohort may be a grouping of athletes based on demographic and/or performance characteristics, such as age, gender, skill level (e.g., professional athlete, amateur athlete, etc.). An athlete may select a cohort similar to themselves for the purpose of comparing their results to similar athletes.

Once P_{pctl} and A_{pctl} for all movements in a movement category have been determined, the P_{pctl} scores may be averaged together and the A_{pctl} scores may be averaged together for every movement in the movement category to achieve a final power and acceleration score for each movement category, denoted P and A (125a-b). Based on P and A, a performance classification for the athlete can be identified per movement category (130a-h). The performance classification is a system for categorizing performance results into insightful groupings which serve as the basis for providing recommendations, and is described in further detail below. The process 100 may be carried out for all movements of a performance test to provide an overall performance classification for the athlete.

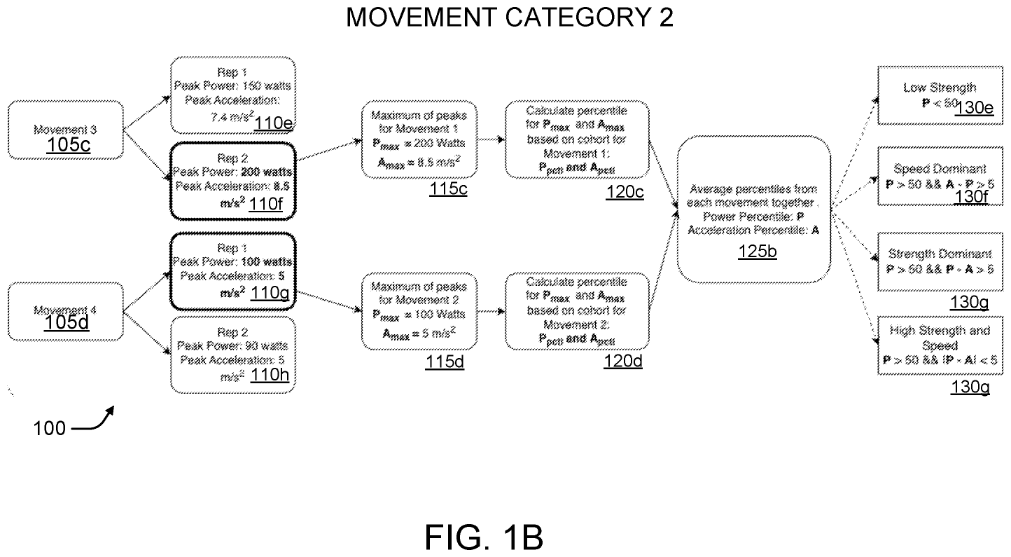

FIG. 2 is a table 200 cross-referencing power and acceleration criteria, performance classifications, and recommended training regimens in one example. The process 100 described above may utilize the table 200 to classify the athlete and generate training recommendations for the athlete. The table 200 illustrates the following classifications:
a) If P<50—Classification: Low Strength
   General Recommendation: Train Strength
b) If P>50 and A−P>5—Classification: Speed Dominant
   General Recommendation: Train Power
c) If P>50 and P−A>5—Classification: Strength Dominant
   General Recommendation: Train Acceleration
d) If P>50 and |P−A|<5—Classification: High Strength and Speed
   General Recommendation: Train Power & Acceleration

Although the table 200 provides general training recommendations for each performance classification, further embodiments can provide specific training routines as described below. Recommendations may be based on percentiles as shown above, which are based on either a specified cohort or all users in example embodiments. Percentiles may be dynamic and may change over time as more performance data is gathered and incorporated into the cohort. Example embodiments can ensure quality recommendations due to curation of tests and movements that align to our power & acceleration based diagnostic algorithms. Athletes may repeat the same test at the same resistance in order to track progress over time.

The use of reference data outside of an athlete's cohort may result in suboptimal recommendations based on percentile (e.g., a 45-year old amateur athlete compared against a cohort to 19-year old college athletes). Even if an athlete's results remain static, their percentile score may change as the cohort includes more data, resulting in potentially different recommendations despite no actual change in the athlete's own performance. Some users may see a plateau or decline in their scores. Further, certain users in very niche cohorts may not be able to generate recommendations until there is more data available.

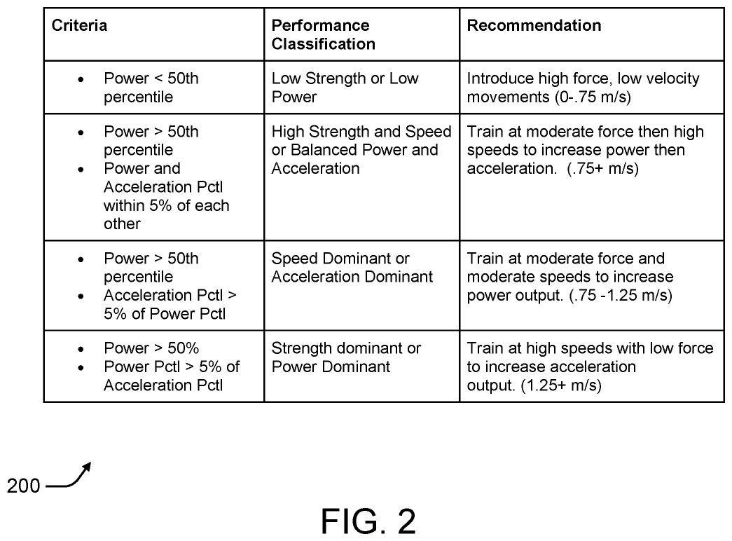

FIGS. 3A-B are tables 300, 301 for interpreting results of elasticity and power balance evaluations in one embodiment. The process 100 described above may utilize the tables 300, 301 to classify the athlete and generate training recommendations for the athlete. Such results can be generated in response to a performance test that includes measurement of the athlete's elasticity of movements via static/elastic movement pair. Based on the percentiles shown in the leftmost column, the athletes may be classified as shown in the middle column, and provided with training recommendations as shown in the rightmost column. For example, if an athlete's elastic is less than 10% higher than the static measurement (e.g., in terms of cohort percentage), then the athlete may be classified as being inefficient as using elasticity to generate power, and may be recommended to incorporate plyometric training into future training sessions. Likewise, if the athlete demonstrates a power imbalance between the muscle groups of the right and left sides of the body, then the athlete may be advised to increase training of the less powerful side.

Example embodiments provide several advantages. For athletic trainers, example embodiments can provide them with a topline understanding of their client's testing results, and supply them with recommendations that they can leverage to adapt and build training programs for their clients. Such insights into their clients can engender greater trust in the trainers, facilitate tracking progression between testing sessions, and provide actionable information with regard to imbalances of strength and speed, power and acceleration, and elasticity. For the athletes, example embodiments can provide a topline understanding of their testing results, and encourage success in their fitness goals by showing progress made between tests. Such embodiments can also provide context to discuss their fitness goals with their trainers and facilitate tracking progression between testing sessions.

Example embodiments can include the following features for assisting a trainer:
a) Finding the right power test to use with a client.
b) Setting a good cohort to generate accurate recommendations.
c) Determining how a client's results compare to their previous test of the same type.
   i. A test is of the same type if it uses the same template/protocol
   ii. Does not apply to “ad-hoc” configured tests
d) Viewing targeted insights for a client
e) Sharing a client's recommendations for viewing away from testing equipment.
f) Understanding how long a client's recommendations are relevant/when they expire.
g) Reporting whether the recommendation was helpful or not.
h) Creating a test that will generate insights & recommendations.
i) Creating a test that is only available for a subset of locations.
j) Tagging movement pairs as “elastic”/“static” pairs.
k) Processing feedback on the efficacy of the recommendations generated.

Further, example embodiment can include the following additional features specific to an athlete's goals:
a) Understand what actions to take based on the generated insights without heavy explanation from a trainer.
b) Generating insights even if the athlete is unable to complete every single testing movement.
c) Displaying recommendations away from the testing equipment.
d) Displaying how current results compare to previous tests.

### Progress Tracking

The process 100 described above may be repeated over time to track an athlete's progress. In one example, after a power-based test is complete, if the athlete has completed the same test previously (e.g., using the same protocol/template):
a) Calculate a power score, acceleration score, and a unilateral imbalance score for each movement category by averaging the scores.
   i. Display a graph showing how results from the last 4 tests compare.
   ii. All data points from previous tests should exactly match the movements completed on the current test, per movement category.
b) Calculate a power score, acceleration score, and a unilateral imbalance score for the entire test by averaging the scores.
   i. Display a graph showing how results from the last 4 tests compare.
   ii. All data points from previous tests must exactly match the movements completed on the current test, per movement category.

### Cohorts and Filtering

In one example, to evaluate an athlete relative to a given cohort (e.g., category of athletes selected by one or more of gender, age, sport, weight class, injury type, performance readiness, or other attributes of the athlete or the sport), a cohort is selected that will be used for generating percentiles. The system may issue a notification if a user has selected a cohort that is very different from themselves. For example:
a) Criteria for displaying warning indicator:
   i. Skill level below user's skill level OR
   ii. Age outside of a 10-year window OR
   iii. Different sex OR
   iv. Different position OR sport

The user interface (UI) may also be configured to prevent a user from generating recommendations based on too small of a cohort. For example, the sample size for movement may be required to be greater than 10 for each movement to be considered for percentile comparisons. Further, the system can utilize existing cohort filtering UI and requirements to 1) prevent users with incomplete profiles from filtering, and 2) Prompt a user with an incomplete profile to complete their profile without navigating away from the session summary.

### Session Summary Results UI

The UI may be configured to display session summary results after a test is complete, which may include:
a) Comparisons Table
b) Insights & Recommendations (if the test was a “power-based test”)
c) Personal Records
d) Leaderboards
e) A diagram (e.g., mannequin) showing what muscles were activated.
f) UI for showing progress relative to previous test of the same type
g) UI that drives users to the other reporting views
   i. Power Report
   ii. Bilateral Balance
   iii. Movement Detail View←→Exercise Detail 3D

### Recommendation UI

For each movement category, the system may display a corresponding recommendation. The recommendations may suggest certain movements to be done on or off given equipment based on a performance classification. Features of this mode may include:
a) Feedback mechanism for whether the recommendations are relevant or not
b) When viewing a test older than 1 month:
   i. Prompt user to re-test with CTA
   ii. Let the user see “expired” recommendations if desired
c) Mechanism for sharing recommendations
d) Display copy to alert trainer/athlete that recommendations are only valid for 1 month
e) Content may be dynamic based on what valid insights and recommendations are available based on the test and the cohort data availability.
   i. Number of insights and recommendations generated may be lower than the maximum number of recommendations.
   ii. Display recommendations if possible, regardless of test completion.

### Sharing Results

Example embodiments may also be configured to provide a UI for sharing recommendations with a user that may not have access to a computer system generating the results. For example, a shared view may show results with certain restrictions, such as by excluding:
a) Cohort filtering UI
b) Feedback mechanism
c) CTA for testing
d) Links to other reporting views
e) Comparisons Table

### Feedback

Example embodiments may be configured to provide an interface (e.g., at a display of a computing device) for reporting if a recommendation is useful or not (e.g. thumbs up/thumbs down, scale 1-5, etc.). The system may store this feedback for future use.

### Example: Evaluating a Baseball Player

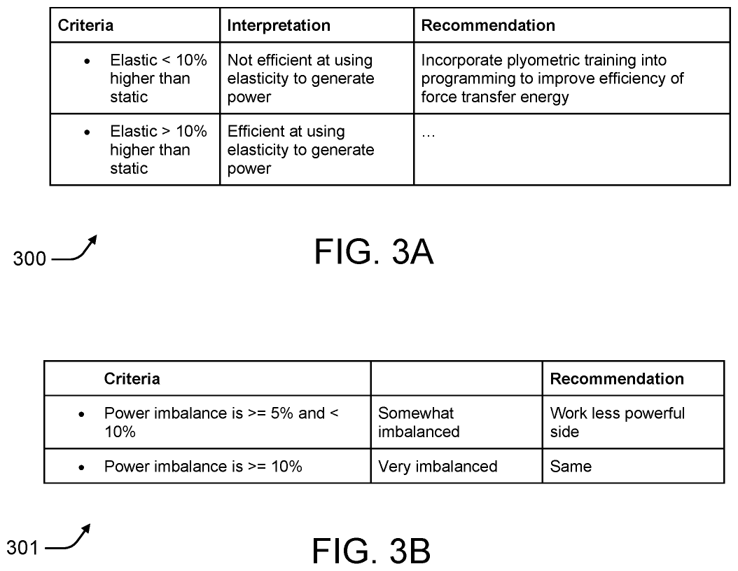

FIGS. 4-9 illustrate an example process of evaluating the athletic performance of an athlete. Although the specific movements and evaluations are relevant to a baseball player, the process may be adapted for an athlete in any sport or other athletic competition. The evaluation process may follow the process 100 described above.

FIG. 4 is a table 400 illustrating an example set of movements performed by the athlete for a performance test that is measured by testing equipment. As shown, the performance test includes 5 distinct movements each performed unilaterally on both sides of the body. Each movement is done 5 times (reps) on each side with a predetermined resistance (e.g., 101bs).

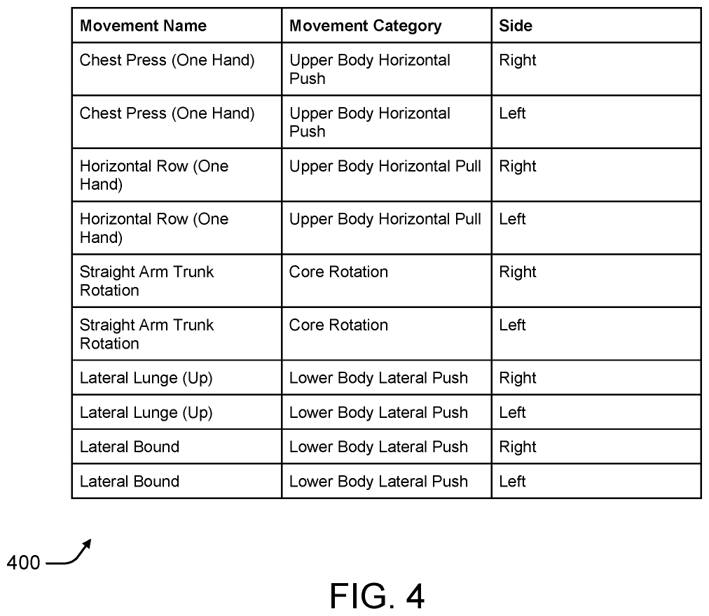

FIG. 5 is a table 500 illustrating movement metrics calculated from position data recorded by the testing equipment during the performance test defined by the table 400. The first column shows the name of each movement, while the remaining columns detail the measured results of the athlete's performance of each movement. In particular, the results may include peak scores of power (Watts) and peak acceleration (m/s 2), along with an indication of which side of the athlete (right/left) performed the movement.

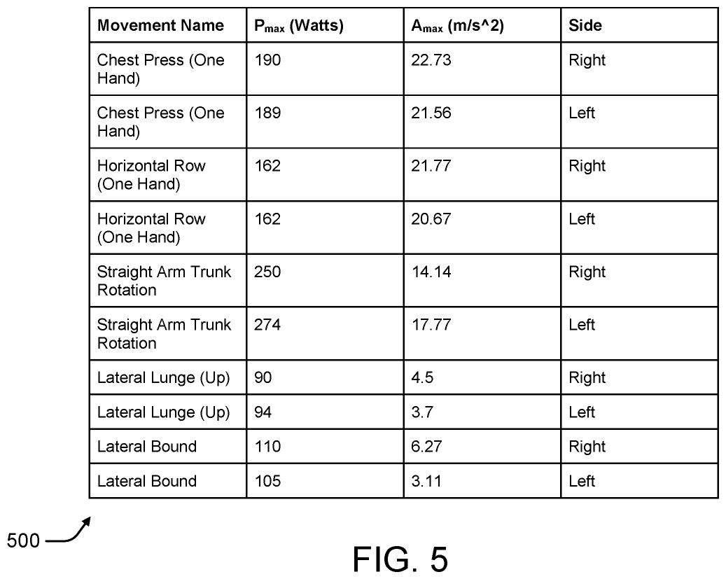

FIG. 6 is a table 600 illustrating percentile scores corresponding to the aforementioned movement data, which result from comparing the movement data against movement data of a selected cohort. The first column shows the name of each movement, while the second and third columns show a percentile score for each peak power and acceleration score compared to other college baseball players (i.e., the athlete's cohort), For example, the athlete's peak scores P_{pctl} and A_{pctl} for the left-side chest press are shown to rank in the 97^{th} percentile for power and the 93^{rd} percentile for acceleration.

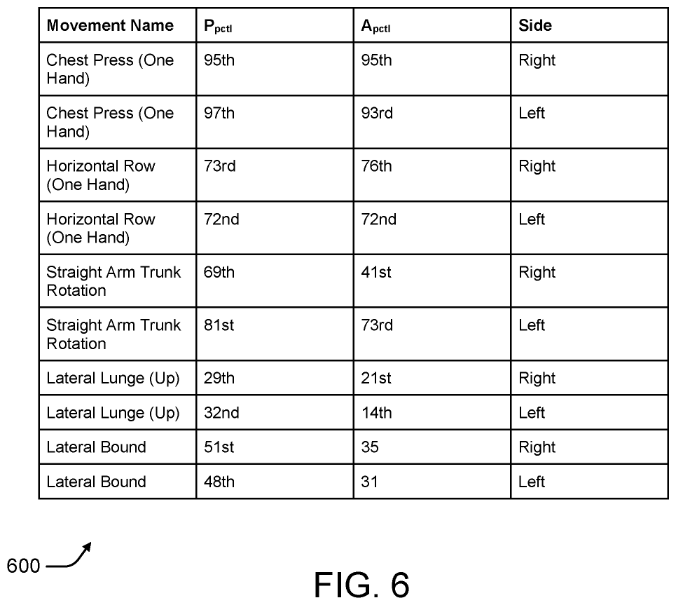

FIG. 7 is a table 700 illustrating averaged percentile scores in one embodiment. To generate this table 700, the unilateral percentile scores of table 600 may be averaged for each movement to achieve the bilateral percentiles shown.

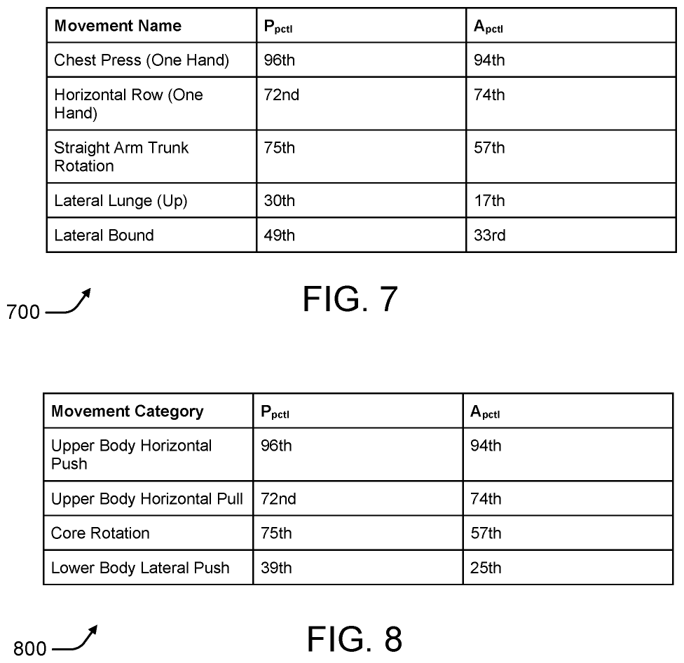

FIG. 8 is a table 800 illustrating movement category percentile scores. Each of the movements of the table 700 may belong to a given movement category. For example, the lateral lunge and lateral bound movements may both belong to the “lower body lateral push” movement category of table 800. Thus, for each movement in a movement category, the P_{pctl} and A_{pctl} scores may be averaged to give a final percentile for each movement category as shown in the table 800.

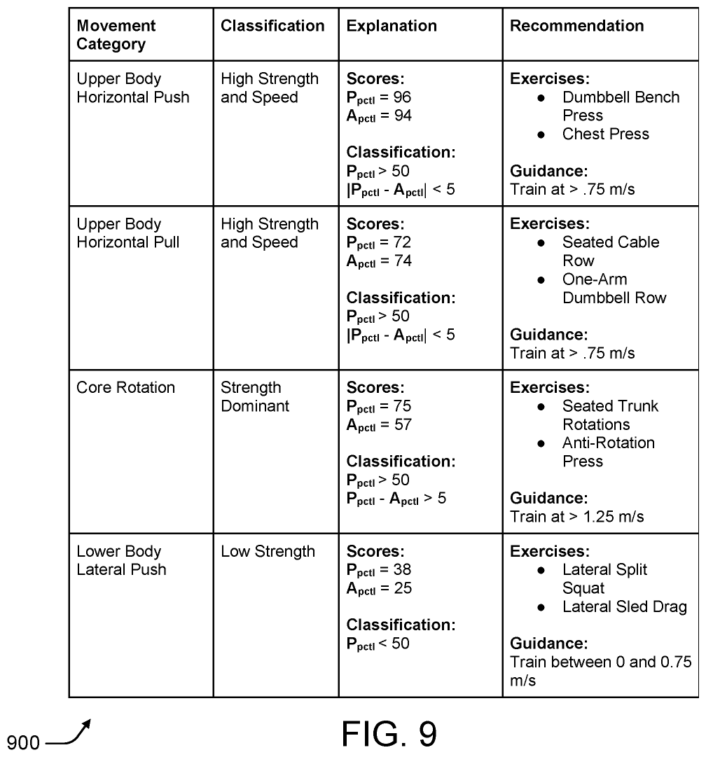

FIG. 9 is a table 900 illustrating athlete performance classification and corresponding training regimen that may be generated based on the movement P_{pctl} and A_{pctl} scores calculated as described above. The first column lists each movement category, while the remaining columns indicate corresponding classifications, explanations of the classification, and recommended training regimens, respectively. Based on the percentiles shown in table 800, the athlete's performance is classified for each movement category. For example, for the “upper body horizontal push” movement category, the P_{pctl} and A_{pctl} scores indicate a classification of “high strength and speed.” The requirements for this classification are indicated in the explanation column: the classification corresponds to P_{pctl} being above 50% and the difference between P_{pctl} and A_{pcfl} being less than 5%. Lastly, the leftmost column indicates a recommended training regimen for the given movement category and the athlete's classification. In this example, the athlete is guided to perform two exercises (dumbbell bench press and chest press) and is guided to perform those exercises at a speed above 0.75 m/s. Example embodiments may display, at a user interface or other device, some or all of the information of the table 900 to inform the athlete of their scores and recommended training regimen.

In contrast, for the movement category of core rotation, the athlete is classified as being “strength dominant,” meaning that the athlete possesses proportionally greater strength than speed for the given movement category. The requirement for this classification, as shown in the table 900, is P_{pctl} being above 50% and the difference between P_{pctl} and A_{pctl} being greater than 5%. Accordingly, the table provides a recommended training regimen for core rotation that includes two exercises (seated trunk rotations and anti-rotation press) and guidance to perform those exercises at a greater speed (i.e., 1.25 m/s) to improve the athlete's speed relative to strength. For the movement category of lower body lateral push, the athlete is classified as being “low strength,” which is met when is P_{pctl} is below 50% regardless of A_{pctl}. Accordingly, the table provides a recommended training regimen for lower body lateral push that includes two exercises (lateral split squat and lateral sled drag) and guidance to perform those exercises at a slower speed (i.e., 1.25 m/s) to improve the athlete's strength.

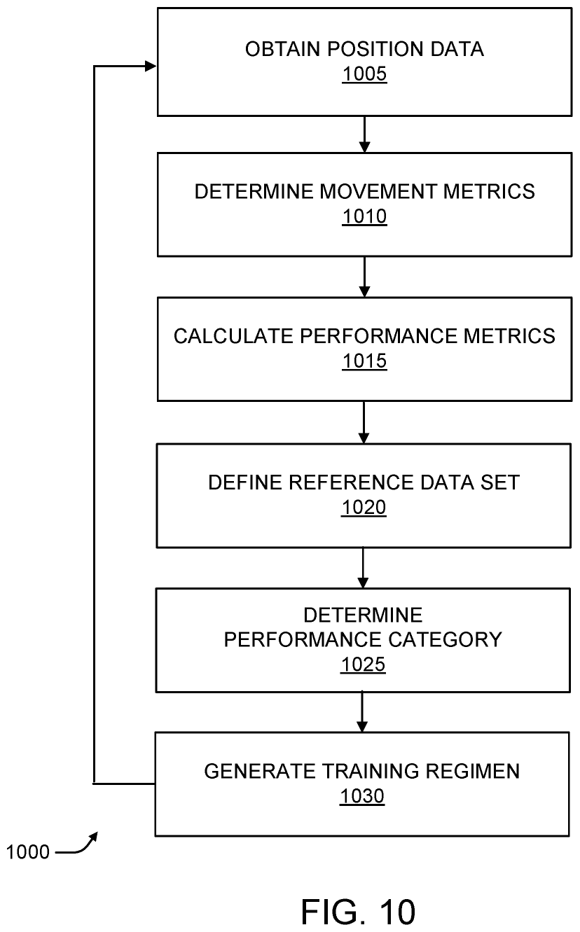

FIG. 10 is a flow diagram of a process 1000 of evaluating athletic performance in one embodiment. The process may include some or all features of the processes described above with reference to FIGS. 1-9. Position data of the athlete during performance of a set of movements may be obtained, the position data indicating position of the athlete over time during the performance of the set of movements (1005). Movement metrics for the set of movements may be determined based on the position data, the movement metrics including measures of acceleration and power (1010). Performance metrics for the athlete may then be calculated based on the movement metrics, the performance metrics including strength and speed (or power and acceleration) (1015). A reference data set may be defined based on a plurality of attributes associated with the athlete (1020). The performance metrics may then be applied to the reference data set to determine a performance category for the athlete, the performance category indicating relative strength and speed of the athlete among other athletes represented in the reference data set (1025). Lastly, a training regimen for the athlete may be generated based on the performance category (1030).

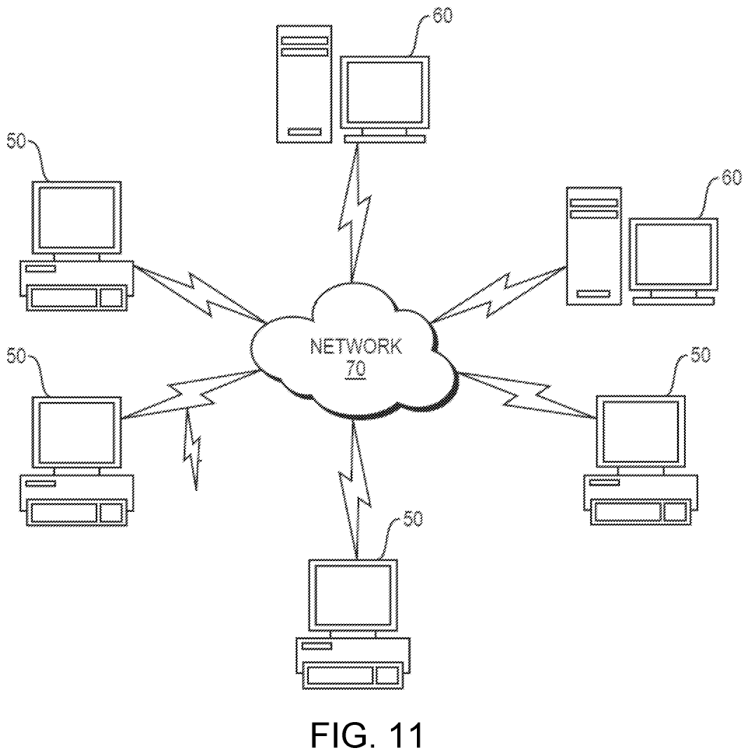

FIG. 11 illustrates a computer network or similar digital processing environment in which embodiments of the present invention may be implemented. Client computer(s)/devices 50 and server computer(s) 60 provide processing, storage, and input/output devices executing application programs and the like, and may be configured to operate some or all features of the processes 100, 1000 described above. The client computer(s)/devices 50 can also be linked through communications network 70 to other computing devices, including other client devices/processes 50 and server computer(s) 60. The communications network 70 can be part of a remote access network, a global network (e.g., the Internet), a worldwide collection of computers, local area or wide area networks, and gateways that currently use respective protocols (TCP/IP, Bluetooth®, etc.) to communicate with one another. Other electronic device/computer network architectures are suitable.

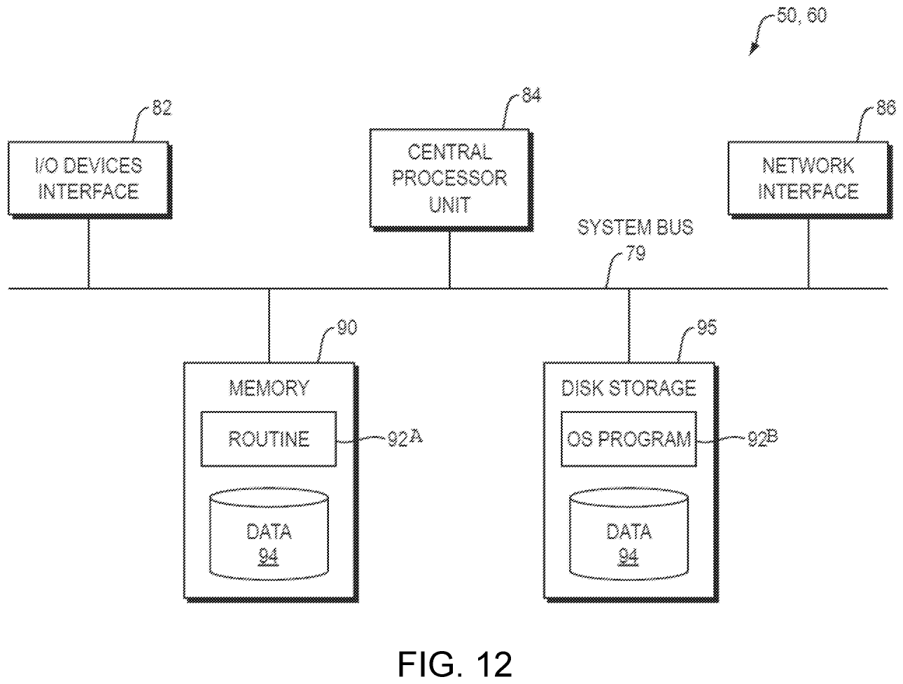

FIG. 12 is a diagram of an example internal structure of a computer (e.g., client processor/device 50 or server computers 60) in the computer system of FIG. 5. Each computer 50, 60 contains a system bus 79, where a bus is a set of hardware lines used for data transfer among the components of a computer or processing system. The system bus 79 is essentially a shared conduit that connects different elements of a computer system (e.g., processor, disk storage, memory, input/output ports, network ports, etc.) that enables the transfer of information between the elements. Attached to the system bus 79 is an I/O device interface 82 for connecting various input and output devices (e.g., keyboard, mouse, displays, printers, speakers, etc.) to the computer 50, 60. A network interface 86 allows the computer to connect to various other devices attached to a network (e.g., network 70 of FIG. 5). Memory 90 provides volatile storage for computer software instructions 92 and data 94 used to implement an embodiment of the present invention (e.g., structure generation module, computation module, and combination module code detailed above). Disk storage 95 provides non-volatile storage for computer software instructions 92 and data 94 used to implement an embodiment of the present invention. A central processor unit 84 is also attached to the system bus 79 and provides for the execution of computer instructions.

In one embodiment, the processor routines 92 and data 94 are a computer program product (generally referenced 92), including a non-transitory computer-readable medium (e.g., a removable storage medium such as one or more DVD-ROM's, CD-ROM's, diskettes, tapes, etc.) that provides at least a portion of the software instructions for the invention system. The computer program product 92 can be installed by any suitable software installation procedure, as is well known in the art. In another embodiment, at least a portion of the software instructions may also be downloaded over a cable communication and/or wireless connection.

While example embodiments have been particularly shown and described, it will be understood by those skilled in the art that various changes in form and details may be made therein without departing from the scope of the embodiments encompassed by the appended claims.

What is claimed is:

1. A method of evaluating an athlete, comprising:
obtaining position data of the athlete during performance of a set of movements, the position data indicating position of the athlete over time during the performance of the set of movements;
determining movement metrics for the set of movements based on the position data, the movement metrics including measures of acceleration and power;
calculating performance metrics for the athlete based on the movement metrics, the performance metrics including strength and speed;
defining a reference data set based on a plurality of attributes associated with the athlete;
applying the performance metrics to the reference data set to determine a performance category for the athlete, the performance category indicating 1) relative strength and speed of the athlete among other athletes represented in the reference data set, and 2) a degree of balance between the relative strength and speed; and
generating a training regimen for the athlete based on the performance category.

2. The method of claim 1, wherein the plurality of attributes associated with the athlete include at least one of age, gender, weight, sport, sport position, sport skill level, and height.

3. The method of claim 1, wherein defining the reference data set includes selecting a subset of a larger data set based on similarities between attributes associated with the subset and the attributes associated with the athlete.

4. The method of claim 1, further comprising:
determining a subset of the set of movements that exhibit peak performance; and
wherein the performance metrics are based on the subset to the exclusion of movements outside of the subset.

5. The method of claim 1, wherein generating the training regimen includes selecting a set of exercises for the training regimen that are indicated to improve the degree of balance between strength and speed.

6. The method of claim 1, wherein the performance category is selected from a set of categories including at least one of 1) low strength, 2) speed dominant, 3) strength dominant, and 4) high strength and speed.

7. The method of claim 1, wherein the performance category is selected from a set of categories including at least one of 1) low power, 2) acceleration dominant, 3) power dominant, and 4) balanced strength and speed.

8. The method of claim 1, wherein the position data is obtained from a machine applying a resistance opposing the set of movements.

9. The method of claim 1, wherein determining the movement metrics for the set of movements is based on a measured force applied by the athlete during the performance of the set of movements.

10. The method of claim 1, wherein determining the movement metrics for the set of movements is based on measured velocity achieved by the athlete during the performance of the set of movements.

11. The method of claim 1, wherein the training regimen includes instructions defining one or more individualized training sessions configured based on the performance of the set of movements.

12. The method of claim 1, wherein the training regimen includes instructions defining at least one workout, training session, or rehabilitation session.

13. The method of claim 1, wherein the training regimen includes instructions defining at least one of a resistance level, training equipment, number of repetitions, number of sets, velocity range, and frequency for at least one training movement.

14. The method of claim 1, wherein determining the performance category is based on a difference between the performance metrics and the reference data set, the difference being at least one or raw values and percentile values.

15. The method of claim 1, further comprising determining, based on the movement metrics, at least one of 1) an endurance metric indicating a measure of endurance of the athlete, 2) a consistency metric indicating a measure of consistency of movement performed by the athlete, and 3) a range of motion (ROM) metric indicating a measure of mobility or flexibility of the athlete.

16. The method of claim 14, wherein generating the training regimen includes selecting a set of exercises for the training regimen that are indicated to improve at least one of the endurance metric, the consistency metric, and the ROM metric.

17. A method of evaluating an athlete, comprising:
obtaining position data of the athlete during performance of a set of movements, the position data indicating position of the athlete over time during the performance of the set of movements;
determining movement metrics for the set of movements based on the position data, the movement metrics including measures of acceleration and power;
calculating performance metrics for the athlete based on the movement metrics, the performance metrics including acceleration and power;
defining a reference data set based on a plurality of attributes associated with the athlete;
applying the performance metrics to the reference data set to determine a performance category for the athlete, the performance category indicating 1) relative acceleration and power of the athlete among other athletes represented in the reference data set, and 2) a degree of balance between the relative acceleration and power; and
generating a training regimen for the athlete based on the performance category.

18. The method of claim 1, wherein generating the training regimen includes selecting a set of exercises for the training regimen that are indicated to improve the degree of balance between acceleration and power.

19. The method of claim 1, wherein the performance category is selected from a set of categories including at least one of 1) low power, 2) acceleration dominant, 3) power dominant, and 4) balanced strength and speed.
